(19) United States
(12) Patent Application Publication
(10) Pub. No.: US 2025/0377349 A1
(43) Pub. Date: Dec. 11, 2025

(54) SYSTEM AND METHOD FOR RAPIDLY TESTING SOIL WATER CHARACTERISTIC CURVE AND SOIL FREEZING CHARACTERISTIC CURVE

(71) Applicant: Harbin Institute of Technology, Harbin (CN)

(72) Inventors: Junling SI, Harbin (CN); Hao ZHENG, Harbin (CN); Daoju REN, Harbin (CN); Shaobing FAN, Harbin (CN); Shanpeng LIU, Harbin (CN); Chongqian MA, Harbin (CN); Tianxing SUN, Harbin (CN)

(73) Assignee: Harbin Institute of Technology, Harbin (CN)

(21) Appl. No.: 19/201,981

(22) Filed: May 8, 2025

(30) Foreign Application Priority Data
Jun. 5, 2024 (CN) 202410723985.4

(51) Int. Cl. G01N 33/24; G01N 1/38; G01N 3/02; G01N 15/08

(52) U.S. Cl. CPC G01N 33/246; G01N 1/38; G01N 3/02; G01N 15/08

(57) ABSTRACT

Provided is a system and method for rapidly testing a soil water characteristic curve and a soil freezing characteristic curve. The system includes a pressure chamber, an air pressure loading system, a temperature control system, a water volume measurement system, and a data acquisition system. The pressure chamber includes a metal mold, a base, a cover plate, and a high-air-entry terracotta panel. The air pressure loading system includes a manometer and an electronic pressure controller. The temperature control system includes a low-temperature thermostatic water bath and a silicone hose. The data acquisition system includes a temperature sensor, a pore pressure transducer, a data acquisition unit, and a computer. The system and method can test both a soil water characteristic curve and a soil freezing characteristic curve of a sample.

### CROSS REFERENCE TO THE RELATED APPLICATIONS

This application is based upon and claims priority to Chinese Patent Application No. 202410723985.4, filed on Jun. 5, 2024, the entire contents of which are incorporated herein by reference.

### TECHNICAL FIELD

The present disclosure belongs to the technical field of geotechnical engineering, and relates to a system and method for rapidly testing a soil water characteristic curve and a soil freezing characteristic curve.

### BACKGROUND

A subgrade serves as the foundation of a road structure. The stability of a subgrade is of great significance to the service life of the whole road and the safe driving of vehicles. A humidity state of a subgrade is an important factor affecting the mechanical analysis and long-term performance of the subgrade. During the actual service of a subgrade, a humidity state of the subgrade is constantly changing. A large number of engineering practices have shown that subgrades are often in an unsaturated state when in service. Therefore, it is necessary to investigate the mechanical properties and long-term performance evolution of subgrades in unsaturated states.

A soil water characteristic curve describes the relationship between matric suction (pore water potentials) and water contents or saturation degrees in soil, and can be used to characterize the water retention of soil, estimate the permeability of unsaturated soil, etc. Currently, a soil water characteristic curve is commonly tested by the pressure plate method based on axis translation. In the pressure plate method, the matric suction in soil is changed by adjusting a pore air pressure, and after the equilibrium of matric suction in soil, a volume of water discharged from a sample is measured. Then, in combination with the volumetric water contents of saturated samples that are measured before testing, the volumetric water contents at different suction levels after equilibrium can be calculated. However, in the pressure plate method, only the discrete data of suction of a sample under several different water content states can be acquired and fitted to produce a continuous soil water characteristic curve, but a continuous soil water characteristic curve of a soil sample cannot be directly acquired. Moreover, the equilibrium of matric suction in a sample requires a lot of time, and it typically takes several hours or even days for a single data point, resulting in relatively low testing efficiency. In seasonally frozen soil regions, subgrades undergo freeze-thaw cycles, and the temperature variations, the generation of ice lenses, and the water migration significantly alter the soil structures, which inevitably affects the water retention of soil structures. In the existing techniques, samples are placed in a thermostatic chamber and subjected to freeze-thaw cycles, and then tested for a soil water characteristic curve, which is complicated and may compromise the test accuracy.

A soil freezing characteristic curve describes the relationship between sub-zero temperatures and unfrozen water contents in soil, and determines the hydraulic and mechanical properties of frozen soil. At present, a soil freezing characteristic curve is often determined by the combination of a low-temperature thermostatic bath and a nuclear magnetic resonance (NMR) system. In this method, the free induction decay of hydrogen nuclei in a magnetic field is measured, and an unfrozen water content is calculated based on the proportional relationship between signal intensities and liquid water. This method has high accuracy and high testing speed, but involves cumbersome testing steps and difficult operations and requires an operator to possess advanced theoretical knowledge of electromagnetism. In addition, this method can only measure an unfrozen water content at a specific temperature and cannot lead to a continuous soil freezing characteristic curve.

### SUMMARY

The present disclosure provides a system and method for rapidly testing a soil water characteristic curve and a soil freezing characteristic curve. The present disclosure is intended to solve the problem that the current soil water characteristic curve testing has low efficiency and cannot directly lead to a continuous soil water characteristic curve of a soil sample and solve the problem that the testing of a soil water characteristic curve after a freeze-thaw cycle involves cumbersome operations and cannot lead to a continuous soil freezing characteristic curve. Moreover, the present disclosure can test both a soil water characteristic curve and a soil freezing characteristic curve of a sample.

The objective of the present disclosure is achieved through the following technical solutions:
A system for rapidly testing a soil water characteristic curve and a soil freezing characteristic curve is provided, including a pressure chamber, an air pressure loading system, a temperature control system, a water volume measurement system, and a data acquisition system,
where the pressure chamber includes a metal mold, a base, a cover plate, and a high-air-entry terracotta panel;
a top of the metal mold is removably connected to the cover plate, and a bottom of the metal mold is removably connected to the base;
the pressure chamber is connected to an output end of the air pressure loading system through an air inlet/outlet port in the cover plate, and is configured to control an air pressure in the pressure chamber;
a water discharge hole is formed at a center of the base, and the base is internally embedded with the high-air-entry terracotta panel matching an inner diameter of the pressure chamber;
the air pressure loading system includes a manometer and an electronic pressure controller, and the manometer is connected to the electronic pressure controller; the electronic pressure controller is connected to a pressure regulator, and the electronic pressure controller is configured to regulate an air pressure value at a specified rate through the pressure regulator to control an air pressure in the metal mold; and the manometer is configured to measure the air pressure in the metal mold;
the water volume measurement system is connected to the water discharge hole, and is configured to measure a water volume change;
the temperature control system includes a low-temperature thermostatic water bath and a silicone hose; and the low-temperature thermostatic water bath is connected to side walls of the metal mold through the silicone hose, such that a coolant circulates in the side walls of the metal mold to achieve a freeze-thaw cycle for a sample; and
the data acquisition system is configured to acquire environmental information inside the sample, and includes a temperature sensor, a pore pressure transducer, a data acquisition unit, and a computer; the data acquisition unit is connected to the temperature sensor, the pore pressure transducer, and the computer; the temperature sensor and the pore pressure transducer are connected to an interior of the pressure chamber through through holes in the cover plate to monitor a temperature and a pore water pressure of the sample in real time, respectively; and the data acquisition unit is configured to transmit the temperature and the pore water pressure of the sample acquired in real time to the computer.

A method for rapidly testing a soil water characteristic curve and a soil freezing characteristic curve is provided, including the following steps:
step 1, saturation of a sample and the high-air-entry terracotta panel:
step 1.1, determining an optimal water content and a maximum dry density of the sample through a compaction test; calculating a dry density under a specified compaction degree, and weighing a mass of a dry soil required for compacting to a specified volume at the dry density; and compacting the sample to the specified volume in layers according to the optimal water content, such that the sample is close to the high-air-entry terracotta panel at the base and four walls of the metal mold;
step 1.2, placing the base and the metal mold in a vacuum saturation device for degassing, and evacuating air in the vacuum saturation device to form a low-pressure or vacuum state inside the vacuum saturation device, where the air in the vacuum saturation device is removed to prevent distilled water from penetrating into pores of the sample subsequently to produce bubbles; opening a knob at a lower side of the vacuum saturation device, and when the metal mold is submerged by the distilled water, closing the knob; and in order to avoid a negative pressure at a side of the high-air-entry terracotta panel, conducting a suction operation from the side of the high-air-entry terracotta panel to ensure a pressure equilibrium between two sides of the high-air-entry terracotta panel to prevent the high-air-entry terracotta panel from being damaged or affecting a saturation process of the sample due to an uneven pressure;
step 1.3, after the distilled water is fully degassed, stopping the air evacuation, and applying an air pressure of about 100 kPa to the pressure chamber; and after the air pressure is applied, continuing the suction operation from the side of the high-air-entry terracotta panel to evacuate air in the sample, such that the distilled water is able to fully penetrate into the pores of the sample to allow a completely saturated state;
step 1.4, after the saturation is completed, measuring a total mass of the sample;
step 1.5, connecting the cover plate to the top of the metal mold, and sealing with the sealing ring; and
step 1.6, inserting the temperature sensor and the pore pressure transducer into the sample at center positions;
step 2, water desorption and water absorption:
step 2.1, the water desorption: increasing an air pressure value at a specified rate by the electronic pressure controller through the pressure regulator to increase matric suction in the sample, such that, as the matric suction increases, water in the sample gradually infiltrates downwards and is discharged out of the sample, passes through the high-air-entry terracotta panel, and is fed into the water volume measurement system through the water discharge hole; and measuring a volume of water discharged from the sample with the water volume measurement system;
step 2.2, the water absorption: decreasing an air pressure value at a specified rate by the electronic pressure controller through the pressure regulator to decrease matric suction in the sample, such that, as the matric suction decreases, water is sucked from the water volume measurement system through the water discharge hole, passes through the high-air-entry terracotta panel, and is absorbed by the sample; and measuring a volume of water absorbed by the sample with the water volume measurement system;
step 2.3, acquiring a value of the pore pressure transducer during testing by the data acquisition unit in real time, such that a pore water pressure in the sample is able to be measured in real time and thus matric suction in the sample is able to be acquired in real time, where the matric suction is produced by subtracting the pore water pressure from a pore air pressure, and there is no need to wait for a suction equilibrium;
step 2.4, according to the total mass of the saturated sample, the mass of the dry soil, and the volume of water discharged from the sample during the testing that are measured in the step 1, calculating mass water contents under different suction levels with the following calculation formula:

$$ω = \frac{{m}_{wet} - {m}_{dry}}{{m}_{dry}} \times 100 \%$$

where m_{wet} represents a mass of a wet soil, m_{dry} represents the mass of the dry soil, and ω represents a mass water content;
step 2.5, calculating volumetric water contents under different suction levels with the following formula:

$${θ}_{w} = {ρ}_{d} \times ω$$

where θ_{w} represents a volumetric water content and ρ_{d} represents the dry density of the sample;
step 2.6, pressurizing at a set rate to a target value, and when a value of the water volume measurement system does not change, indicating that the sample reaches a water desorption equilibrium; and with a matric suction level as an x-coordinate, namely a logarithmic coordinate, and a volumetric water content as a y-coordinate, plotting a soil water characteristic curve for the water desorption of the sample through Van Genuchten or Fredlund-Xing model fitting; and
step 2.7, depressurizing at a set rate to a target value, and when a value of the water volume measurement system does not change, indicating that the sample reaches a water absorption equilibrium; and with a matric suction level as an x-coordinate, namely a logarithmic coordinate, and a volumetric water content as a y-coordinate, plotting a soil water characteristic curve for the water absorption of the sample through Van Genuchten or Fredlund-Xing model fitting;
step 3, soil freezing characteristic curve testing:
step 3.1, saturating the sample and the high-air-entry terracotta panel according to the step 1; and freezing the sample with the low-temperature thermostatic water bath, and when a value of the pore pressure transducer does not change, stopping the freezing; and
step 3.2, acquiring values of the temperature sensor and the pore pressure transducer during testing by the data acquisition unit in real time, and calculating an unfrozen water content in the sample at a current temperature according to the soil water characteristic curves determined in the step 2; and with a temperature as an x-coordinate and an unfrozen water content of the sample as a y-coordinate, plotting a soil freezing characteristic curve of the sample;
step 4, soil water characteristic curve testing after a freeze-thaw cycle:
step 4.1, freezing the sample according to the step 3.1; and
step 4.2, after the freezing is completed, thawing the sample, and testing a soil water characteristic curve for the sample after the freeze-thaw cycle according to the step 2.

Compared with the prior art, the present disclosure has the following advantages:
1. In the traditional method, the equilibrium of matric suction in a sample requires a lot of time. Moreover, the traditional method can only acquire the discrete data of suction of a sample under several different water content states, and cannot directly lead to a continuous soil water characteristic curve of a soil sample. In the continuous pressurization method based on axis translation proposed by the present disclosure, a pore pressure transducer is placed in a sample, such that a pore water pressure in the sample can be measured in real time and thus the matric suction in the sample can be acquired in real time, without a need to wait for a suction equilibrium. As a result, the method of the present disclosure can greatly improve the efficiency of testing a soil water characteristic curve, and can directly lead to a continuous soil water characteristic curve of a soil sample. In addition, the present disclosure adopts a low-temperature thermostatic water bath to directly freeze and thaw the sample, such that the testing of a soil water characteristic curve after a freeze-thaw cycle can be achieved, which involves convenient operations and significantly improves the measurement accuracy.
2. During a freezing process of the low-temperature thermostatic water bath in the present disclosure, the current unfrozen water content can be converted from the measured matric suction in a sample according to a soil water characteristic curve. The current temperature can be acquired by a temperature sensor in the sample. Accordingly, a continuous soil freezing characteristic curve of the sample can be plotted based on temperatures and unfrozen water contents. The present disclosure has various functions, and can test both a soil water characteristic curve and a soil freezing characteristic curve of a sample.

### BRIEF DESCRIPTION OF THE DRAWINGS

FIG. 1 is a schematic diagram of a structure of the system for rapidly testing a soil water characteristic curve and a soil freezing characteristic curve; and

FIG. 2 is a partial enlarged view of FIG. 1.

Reference numerals: 1—computer, 2—data acquisition unit, 3—low-temperature thermostatic water bath, 4—temperature sensor, 5—pressure regulator, 6—electronic pressure controller, 7—double-tube burette, 8—differential pressure gauge, 9—soil sample, 10—pore pressure transducer, 11—manometer, 12—porous probe, 13—terracotta panel, 14—cover plate, 15—base, and 16—switch knob.

### DETAILED DESCRIPTION OF THE EMBODIMENTS

The technical solutions of the present disclosure are further described below with reference to the accompanying drawings, but the present disclosure is not limited thereto. Any modification or equivalent replacement made to the technical solutions of the present disclosure without departing from the spirit and scope of the technical solutions of the present disclosure should fall within the protection scope of the present disclosure.

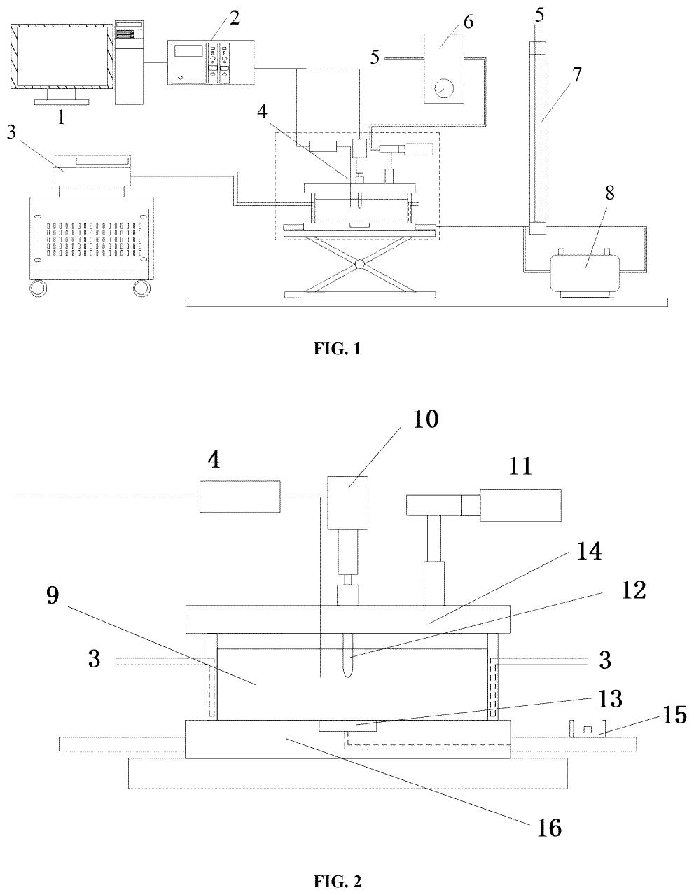

The present disclosure provides a system for rapidly testing a soil water characteristic curve and a soil freezing characteristic curve, as shown in FIG. 1 and FIG. 2. The system includes a pressure chamber, an air pressure loading system, a temperature control system, a water volume measurement system, and a data acquisition system.

The pressure chamber includes a metal mold, a base, a cover plate, a high-air-entry terracotta panel, and sealing rings.

A top of the metal mold is removably connected to the cover plate, and a bottom of the metal mold is removably connected to the base. The sealing rings are arranged for sealing to prevent a pressure loss in the pressure chamber due to air leakage.

The pressure chamber is connected to an output end of the air pressure loading system through an air inlet/outlet port in the cover plate, and is configured to control an air pressure in the pressure chamber.

A water discharge hole is formed at a center of the base, and the base is internally embedded with the high-air-entry terracotta panel matching an inner diameter of the pressure chamber.

The air pressure loading system includes a manometer and an electronic pressure controller, and the manometer is connected to the electronic pressure controller. The electronic pressure controller is connected to a pressure regulator, and the electronic pressure controller is configured to regulate an air pressure value at a specified rate through the pressure regulator to control an air pressure in the metal mold. The manometer is configured to measure the air pressure in the metal mold.

The water volume measurement system is connected to the water discharge hole, and is configured to measure a water volume change, which can be achieved in various manners. The following two solutions are listed:
Solution 1: The water volume measurement system includes a differential pressure gauge and a double-tube burette. The differential pressure gauge is connected to the pressure regulator. The double-tube burette is connected to the water discharge hole through a conduit to allow a water discharge during testing. The differential pressure gauge is connected to the double-tube burette to measure a discharged water volume.
Solution 2: The water volume measurement system includes a laser displacement sensor, a double-tube burette, and a float ball. The double-tube burette is connected to the water discharge hole through a conduit to allow a water discharge during testing. The float ball is placed in the double-tube burette. A position change of the float ball is acquired by the laser displacement sensor to measure the water volume change.

The temperature control system includes a low-temperature thermostatic water bath and a silicone hose. The low-temperature thermostatic water bath is connected to side walls of the metal mold through the silicone hose, such that a coolant circulates in the side walls of the metal mold to achieve a freeze-thaw cycle for a sample.

The data acquisition system is configured to acquire environmental information inside the sample, and includes a temperature sensor, a pore pressure transducer, a data acquisition unit, and a computer. The data acquisition unit is connected to the temperature sensor, the pore pressure transducer, and the computer. Through holes are formed in the cover plate. The temperature sensor and the pore pressure transducer are connected to an interior of the pressure chamber through the through holes in the cover plate to monitor a temperature and a pore water pressure of the sample in real time, respectively. The data acquisition unit is configured to transmit the temperature and the pore water pressure of the sample acquired in real time to the computer.

A method for rapidly testing a soil water characteristic curve and a soil freezing characteristic curve is provided, including the following steps:
Step 1: Saturation of a sample and the high-air-entry terracotta panel:
Step 1.1: An optimal water content and a maximum dry density of the sample are determined through a compaction test. A dry density under a specified compaction degree is calculated, and a mass of a dry soil required for compacting to a specified volume at the dry density is weighed. The sample is compacted to the specified volume in layers according to the optimal water content, such that the sample is close to the high-air-entry terracotta panel at the base and four walls of the metal mold.
Step 1.2: The base and the metal mold are placed in a vacuum saturation device for degassing, and air in the vacuum saturation device is evacuated to form a low-pressure or vacuum state inside the vacuum saturation device. The air in the vacuum saturation device is removed to prevent distilled water from penetrating into pores of the sample subsequently to produce bubbles. A knob at a lower side of the vacuum saturation device is opened, and when the metal mold is submerged by the distilled water, the knob is closed. In order to avoid a negative pressure at a side of the high-air-entry terracotta panel, a suction operation is conducted from the side of the high-air-entry terracotta panel to ensure a pressure equilibrium between two sides of the high-air-entry terracotta panel to prevent the high-air-entry terracotta panel from being damaged or affecting a saturation process of the sample due to an uneven pressure.
Step 1.3: After the distilled water is fully degassed, the air evacuation is stopped, and an air pressure of about 100 kPa is applied to the pressure chamber. After the air pressure is applied, the suction operation is continued from the side of the high-air-entry terracotta panel to evacuate air in the sample, such that the distilled water can fully penetrate into the pores of the sample to allow a completely saturated state.
Step 1.4: After the saturation is completed, a total mass of the sample is measured.
Step 1.5: The cover plate is connected to the top of the metal mold, and sealing is conducted with the sealing ring.
Step 1.6: The temperature sensor and the pore pressure transducer are inserted into the sample at center positions.
Step 2: Water desorption and water absorption:
Step 2.1: Water desorption: An air pressure value is increased at a specified rate by the electronic pressure controller through the pressure regulator to increase matric suction in the sample. As the matric suction increases, water in the sample gradually infiltrates downwards and is discharged out of the sample, passes through the high-air-entry terracotta panel, and is fed into the double-tube burette through the conduit. A volume of water discharged from the sample is measured with the water volume measurement system.
Step 2.2: Water absorption: An air pressure value is decreased at a specified rate by the electronic pressure controller through the pressure regulator to decrease matric suction in the sample. As the matric suction decreases, water is sucked from the double-tube burette through the conduit, passes through the high-air-entry terracotta panel, and is absorbed by the sample. A volume of water absorbed by the sample can be measured with the water volume measurement system.
Step 2.3: A value of the pore pressure transducer during testing is acquired by the data acquisition unit in real time, such that a pore water pressure in the sample can be measured in real time and thus matric suction in the sample can be acquired in real time (the matric suction is produced by subtracting the pore water pressure from a pore air pressure), without a need to wait for a suction equilibrium.
Step 2.4: According to the total mass of the saturated sample, the mass of the dry soil, and the volume of water discharged from the sample during the testing that are measured in the step 1, mass water contents under different suction levels can be calculated with the following calculation formula:

$$ω = \frac{{m}_{wet} - {m}_{dry}}{{m}_{dry}} \times 100 \%$$

where m_{wet} represents a mass of a wet soil, m_{dry} represents the mass of the dry soil, and ω represents a mass water content.
Step 2.5: Volumetric water contents under different suction levels can be calculated with the following formula:

$${θ}_{w} = {ρ}_{d} \times ω$$

where θ_{w} represents a volumetric water content and ρ_{d} represents the dry density of the sample.
Step 2.6: Water desorption: Pressurization is conducted at a set rate to a target value. When a value of the water volume measurement system does not change, it indicates that the sample reaches a water desorption equilibrium. With a matric suction level as an x-coordinate, namely a logarithmic coordinate, and a volumetric water content as a y-coordinate, a soil water characteristic curve for the water desorption of the sample is plotted through Van Genuchten or Fredlund-Xing model fitting.
Step 2.7: Water absorption: Depressurization is conducted at a set rate to a target value. When a value of the water volume measurement system does not change, it indicates that the sample reaches a water absorption equilibrium. With a matric suction level as an x-coordinate, namely a logarithmic coordinate, and a volumetric water content as a y-coordinate, a soil water characteristic curve for the water absorption of the sample is plotted through Van Genuchten or Fredlund-Xing model fitting.
Step 3: Soil freezing characteristic curve testing:
Step 3.1: The sample and the high-air-entry terracotta panel are saturated according to the step 1. The sample is frozen with the low-temperature thermostatic water bath. When a value of the pore pressure transducer does not change, the freezing is stopped.
Step 3.2: Values of the temperature sensor and the pore pressure transducer during testing are acquired by the data acquisition unit in real time, and an unfrozen water content in the sample at a current temperature is calculated according to the soil water characteristic curves determined in the step 2. With a temperature as an x-coordinate and an unfrozen water content of the sample as a y-coordinate, a soil freezing characteristic curve of the sample is plotted.
Step 4: Soil water characteristic curve testing after a freeze-thaw cycle:
Step 4.1: The sample is frozen according to the step 3.1.
Step 4.2: After the freezing is completed, the sample is thawed, and soil water characteristic curves for the sample after the freeze-thaw cycle can be tested according to the step 2.

What is claimed is:

1. A system for rapidly testing a soil water characteristic curve and a soil freezing characteristic curve, comprising a pressure chamber, an air pressure loading system, a temperature control system, a water volume measurement system, and a data acquisition system,
wherein the pressure chamber comprises a metal mold, a base, a cover plate, and a high-air-entry terracotta panel;
a top of the metal mold is removably connected to the cover plate, and a bottom of the metal mold is removably connected to the base;
the pressure chamber is connected to an output end of the air pressure loading system through an air inlet/outlet port in the cover plate, and is configured to control an air pressure in the pressure chamber;
a water discharge hole is formed at a center of the base, and the base is internally embedded with the high-air-entry terracotta panel matching an inner diameter of the pressure chamber;
the air pressure loading system comprises a manometer and an electronic pressure controller, and the manometer is connected to the electronic pressure controller; the electronic pressure controller is connected to a pressure regulator, and the electronic pressure controller is configured to regulate an air pressure value at a specified rate through the pressure regulator to control an air pressure in the metal mold; and the manometer is configured to measure the air pressure in the metal mold;
the water volume measurement system is connected to the water discharge hole, and is configured to measure a water volume change;
the temperature control system comprises a low-temperature thermostatic water bath and a silicone hose; and the low-temperature thermostatic water bath is connected to side walls of the metal mold through the silicone hose, whereby a coolant circulates in the side walls of the metal mold to achieve a freeze-thaw cycle for a sample; and
the data acquisition system is configured to acquire environmental information inside the sample, and the data acquisition system comprises a temperature sensor, a pore pressure transducer, a data acquisition unit, and a computer; the data acquisition unit is connected to the temperature sensor, the pore pressure transducer, and the computer; the temperature sensor and the pore pressure transducer are connected to an interior of the pressure chamber through through holes in the cover plate to monitor a temperature and a pore water pressure of the sample in real time, respectively; and the data acquisition unit is configured to transmit the temperature and the pore water pressure of the sample acquired in real time to the computer.

2. The system according to claim 1, wherein a sealing ring is provided between the top of the metal mold and the cover plate and between the bottom of the metal mold and the base for sealing.

3. The system according to claim 1, wherein the water volume measurement system comprises a differential pressure gauge and a double-tube burette; the differential pressure gauge is connected to the pressure regulator; the double-tube burette is connected to the water discharge hole through a conduit to allow a water discharge during testing; and the differential pressure gauge is connected to the double-tube burette to measure a discharged water volume.

4. The system according to claim 1, wherein the water volume measurement system comprises a laser displacement sensor, a double-tube burette, and a float ball; the double-tube burette is connected to the water discharge hole through a conduit to allow a water discharge during testing; the float ball is placed in the double-tube burette; and a position change of the float ball is acquired by the laser displacement sensor to measure the water volume change.

5. A method for rapidly testing a soil water characteristic curve and a soil freezing characteristic curve using the system according to claim 1, comprising the following steps:
step 1, saturation of the sample and the high-air-entry terracotta panel:
step 1.1, determining an optimal water content and a maximum dry density of the sample through a compaction test; calculating a dry density under a specified compaction degree, and weighing a mass of a dry soil required for compacting to a specified volume at the dry density; and compacting the sample to the specified volume in layers according to the optimal water content, whereby the sample is tightly close to the high-air-entry terracotta panel at the base and four walls of the metal mold;
step 1.2, placing the base and the metal mold in a vacuum saturation device for degassing, and evacuating air in the vacuum saturation device to form a low-pressure or vacuum state inside the vacuum saturation device, wherein the air in the vacuum saturation device is removed to prevent distilled water from penetrating into pores of the sample subsequently to produce bubbles; opening a knob at a lower side of the vacuum saturation device; and when the metal mold is submerged by the distilled water, closing the knob; wherein in order to avoid a negative pressure at a side of the high-air-entry terracotta panel, a suction operation is conducted from the side of the high-air-entry terracotta panel to ensure a pressure equilibrium between two sides of the high-air-entry terracotta panel to prevent the high-air-entry terracotta panel from being damaged or affecting a saturation process of the sample due to an uneven pressure;
step 1.3, after the distilled water is fully degassed, stopping the air evacuation, and applying an air pressure of about 100 kPa to the pressure chamber; and after the air pressure is applied, continuing the suction operation from the side of the high-air-entry terracotta panel to evacuate air in the sample, whereby the distilled water is allowed to fully penetrate into the pores of the sample to allow a completely saturated state;
step 1.4, after the saturation is completed to obtain a saturated sample, measuring a total mass of the saturated sample;
step 1.5, connecting the cover plate to the top of the metal mold, and sealing with a sealing ring; and
step 1.6, inserting the temperature sensor and the pore pressure transducer into the sample at center positions;
step 2, water desorption and water absorption:
step 2.1, the water desorption: increasing the air pressure value at the specified rate by the electronic pressure controller through the pressure regulator to increase a matric suction in the sample, whereby, as the matric suction increases, water in the sample gradually infiltrates downwards and is discharged out of the sample, passes through the high-air-entry terracotta panel, and is fed into the water volume measurement system through the water discharge hole; and measuring a volume of water discharged from the sample with the water volume measurement system;
step 2.2, the water absorption: decreasing the air pressure value at the specified rate by the electronic pressure controller through the pressure regulator to decrease the matric suction in the sample, whereby, as the matric suction decreases, water is sucked from the water volume measurement system through the water discharge hole, passes through the high-air-entry terracotta panel, and is absorbed by the sample; and measuring a volume of water absorbed by the sample with the water volume measurement system;
step 2.3, acquiring a value of the pore pressure transducer during testing by the data acquisition unit in real time, whereby the pore water pressure in the sample is allowed to be measured in real time and thus the matric suction in the sample is allowed to be acquired in real time, wherein the matric suction is produced by subtracting the pore water pressure from a pore air pressure, and there is no need to wait for a suction equilibrium;
step 2.4, according to the total mass of the saturated sample, the mass of the dry soil, and the volume of water discharged from the sample during testing that are measured in the step 1, calculating mass water contents under different suction levels with the following calculation formula:
$$ω = \frac{{m}_{wet} - {m}_{dry}}{{m}_{dry}} \times 100 \%$$
wherein m_{wet} represents a mass of a wet soil, m_{dry} represents the mass of the dry soil, and w represents a mass water content;
step 2.5, calculating volumetric water contents under different suction levels with the following formula:
$${θ}_{w} = {ρ}_{d} \times ω$$
wherein θ_{w} represents a volumetric water content and ρ_{d} represents the dry density of the sample;
step 2.6, pressurizing at a first set rate to a first target value, and when a value of the water volume measurement system does not change, indicating that the sample reaches a water desorption equilibrium; and with a matric suction level as an x-coordinate, namely a logarithmic coordinate, and a volumetric water content as a y-coordinate, plotting the soil water characteristic curve for the water desorption of the sample through Van Genuchten or Fredlund-Xing model fitting; and
step 2.7, depressurizing at a second set rate to a second target value, and when the value of the water volume measurement system does not change, indicating that the sample reaches a water absorption equilibrium; and with the matric suction level as the x-coordinate, namely the logarithmic coordinate, and the volumetric water content as the y-coordinate, plotting the soil water characteristic curve for the water absorption of the sample through Van Genuchten or Fredlund-Xing model fitting;
step 3, soil freezing characteristic curve testing:
step 3.1, saturating the sample and the high-air-entry terracotta panel according to the step 1; and freezing the sample with the low-temperature thermostatic water bath, and when the value of the pore pressure transducer does not change, stopping the freezing; and
step 3.2, acquiring values of the temperature sensor and the pore pressure transducer during testing by the data acquisition unit in real time, and calculating an unfrozen water content in the sample at a current temperature according to the soil water characteristic curves determined in the step 2; and with the current temperature as the x-coordinate and the unfrozen water content of the sample as the y-coordinate, plotting the soil freezing characteristic curve of the sample; and
step 4, soil water characteristic curve testing after a freeze-thaw cycle:
step 4.1, freezing the sample according to the step 3.1; and
step 4.2, after the freezing is completed, thawing the sample, and testing the soil water characteristic curve for the sample after the freeze-thaw cycle according to the step 2.

6. The method according to claim 5, wherein the sealing ring is provided between the top of the metal mold and the cover plate and between the bottom of the metal mold and the base for sealing.

7. The method according to claim 5, wherein the water volume measurement system comprises a differential pressure gauge and a double-tube burette; the differential pressure gauge is connected to the pressure regulator; the double-tube burette is connected to the water discharge hole through a conduit to allow a water discharge during testing; and the differential pressure gauge is connected to the double-tube burette to measure a discharged water volume.

8. The method according to claim 5, wherein the water volume measurement system comprises a laser displacement sensor, a double-tube burette, and a float ball; the double-tube burette is connected to the water discharge hole through a conduit to allow a water discharge during testing; the float ball is placed in the double-tube burette; and a position change of the float ball is acquired by the laser displacement sensor to measure the water volume change.
